(19) United States
(12) Patent Application Publication
(10) Pub. No.: US 2009/0112221 A1
(43) Pub. Date: Apr. 30, 2009

(54) SYSTEM AND METHOD FOR MEASURING THE SHAPE OF INTERNAL BODY CAVITIES

(75) Inventors: Ronald Burke, Deephaven, MN (US); Benjamin F. Carter, Eden Prairie, MN (US); Alexander Arsenyev, Eagan, MN (US); Jacob John, Eagan, MN (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: Disc Dynamics, Inc., Eden Prairie, MN (US)

(21) Appl. No.: 12/015,359

(22) Filed: Jan. 16, 2008

Related U.S. Application Data
(60) Provisional application No. 60/982,665, filed on Oct. 25, 2007.

(51) Int. Cl. A61B 17/88; A61F 2/46

(52) U.S. Cl. 606/102; 606/78; 623/17.12

(57) ABSTRACT

A method and system of evaluating the geometry of the nuclear cavity within the annulus. A three-dimensional mold of the nuclear cavity is created. The three-dimensional mold is preferably removed from the nuclear cavity. The three-dimensional mold can be used for qualitative and quantitative analysis of the nuclear cavity.

The present application claims the benefit of U.S. Provisional Application Ser. No. 60/982,665 entitled SYSTEM AND METHOD FOR MEASURING THE SHAPE OF INTERNAL BODY CAVITIES, filed on Oct. 25, 2007, which is hereby incorporated by reference.

### FIELD OF THE INVENTION

The present invention relates to systems and methods for evaluating and assessing an internal shape of a body cavity, and for preparing the body cavity to receive a prosthetic device. More particularly, the present invention relates to a method for evaluating a nuclear cavity in an annulus located in an intervertebral disc space and for preparing the nuclear cavity to receive an intervertebral prosthesis.

### BACKGROUND OF THE INVENTION

The intervertebral discs, which are located between adjacent vertebrae in the spine, provide structural support for the spine as well as the distribution of forces exerted on the spinal column. An intervertebral disc consists of three major components: cartilage endplates, nucleus pulpous, and annulus fibrosus. The central portion, the nucleus pulpous or nucleus, is relatively soft and gelatinous; being composed of about 70 to 90% water. The nucleus pulpous has a high proteoglycan content and contains a significant amount of Type II collagen and chondrocytes. Surrounding the nucleus is the annulus fibrosus, which has a more rigid consistency and contains an organized fibrous network of approximately 40% Type I collagen, 60% Type II collagen, and fibroblasts. The annular portion serves to provide peripheral mechanical support to the disc, afford torsional resistance, and contain the softer nucleus while resisting its hydrostatic pressure.

Intervertebral discs, however, are susceptible to a number of injuries. Disc herniation occurs when the nucleus begins to extrude through an opening in the annulus, often to the extent that the herniated material impinges on nerve roots in the spine or spinal cord. The posterior and postero lateral portions of the annulus are most susceptible to attenuation or herniation, and therefore, are more vulnerable to hydrostatic pressures exerted by vertical compressive forces on the intervertebral disc. Various injuries and deterioration of the intervertebral disc and annulus fibrosus are discussed by Osti et al., Annular Tears and Disc Degeneration in the Lumbar Spine, J. Bone and Joint Surgery, 74-B(5), (1982) pp. 678-682; Osti et al., Annulus Tears and Intervertebral Disc Degeneration, Spine, 15(8), (1990) pp. 762-767; Kamblin et al., Development of Degenerative Spondylosis of the Lumbar Spine after Partial Discectomy, Spine, 20(5), (1995) pp. 599-607.

Many treatments for intervertebral disc injury have involved the use of nuclear prostheses or disc spacers. A variety of prosthetic nuclear implants are known in the art. For example, U.S. Pat. No. 5,047,055 (Bao et al.) teaches a swellable hydrogel prosthetic nucleus. Other devices known in the art, such as intervertebral spacers, use wedges between vertebrae to reduce the pressure exerted on the disc by the spine. Intervertebral disc implants for spinal fusion are known in the art as well, such as disclosed in U.S. Pat. No. 5,425,772 (Brantigan) and U.S. Pat. No. 4,834,757 (Brantigan).

Further approaches are directed toward fusion of the adjacent vertebrate, e.g., using a cage in the manner provided by Sulzer. Sulzer's BAK® Interbody Fusion System involves the use of hollow, threaded cylinders that are implanted between two or more vertebrae. The implants are packed with bone graft to facilitate the growth of vertebral bone. Fusion is achieved when adjoining vertebrae grow together through and around the implants, resulting in stabilization.

Apparatuses and/or methods intended for use in disc repair have also been described but none appear to have been further developed, and certainly not to the point of commercialization. See, for instance, French Patent Appl. No. FR 2 639 823 (Garcia) and U.S. Pat. No. 6,187,048 (Milner et al.).

Prosthetic implants formed of biomaterials that can be delivered and cured in situ, using minimally invasive techniques to form a prosthetic nucleus within an intervertebral disc have been described in U.S. Pat. No. 5,556,429 (Felt) and U.S. Pat. No. 5,888,220 (Felt et al.), and U.S. Patent Publication No. US 2003/0195628 (Felt et al.), the disclosures of which are incorporated herein by reference. The disclosed method includes, for instance, the steps of inserting a collapsed mold apparatus (which in a preferred embodiment is described as a “mold”) through an opening within the annulus, and filling the mold to the point that the evaluation material expands with a flowable biomaterial that is adapted to cure in situ and provide a permanent disc replacement. Related methods are disclosed in U.S. Pat. No. 6,224,630 (Bao et al.), entitled “Implantable Tissue Repair Device” and U.S. Pat. No. 6,079,868 (Rydell), entitled “Static Mixer.”

### BRIEF SUMMARY OF THE INVENTION

A nuclectomy is a surgical procedure during which at least a portion of a nucleus material is removed from a disc to create a nuclear cavity in an intervertebral disc space. A nuclectomy is typically performed to prepare a nuclear cavity to receive an intervertebral prosthesis. One embodiment of the present invention relates to a method and system of evaluating the geometry of the nuclear cavity within the annulus. A three-dimensional mold of the nuclear cavity is created in situ. The three-dimensional mold is preferably removed from the nuclear cavity. The three-dimensional mold can be used for qualitative and quantitative analysis of the nuclear cavity. According to various embodiments of the present invention, the three-dimensional mold can be used to evaluate the size and shape of the cavity.

The method includes forming at least one annulotomy in the annulus to provide access to a nucleus. In an embodiment where primary and secondary annulotomies are formed, a separate removal sequence is preferably identified for each of the annulotomies. Nucleus material is removed using one or more surgical tools. Various sequences for removing the nuclear material are disclosed in U.S. Patent Publication No. 2006/0135959, entitled Nuclectomy Method and Apparatus, the complete disclosure of which is incorporated by reference.

In one embodiment, a three-dimensional mold system is used to form a mold in situ in the nuclear cavity. The three-dimensional mold is then preferably removed from the nuclear cavity and evaluated by the surgeon. The three-dimensional mold permits the surgeon to evaluate the internal geometric features of the nuclear cavity and/or to estimate the quantity of nucleus material removed as well as the geometry of the nuclear cavity. For example, the evaluation mold can be used to determine whether all of the targeted nucleus material has been removed, whether the nuclear cavity is centered within the annulus, and/or whether the nuclear cavity is symmetrical relative to the midline of the spine. One or more of the removing steps are optionally repeated as necessary until an adequate amount of the nucleus is removed from the annulus. Other determinations regarding the internal features of the nuclear cavity can also be made. For example, determination regarding the presence of bone spurs or other patient specific bone/endplate anatomy can be made.

The present three-dimensional mold system can be use with or without a balloon. In one embodiment, the evaluation balloon is positioned in the nuclear cavity and a fluid is delivered to the evaluation balloon so that the balloon substantially fills the nuclear cavity. A method and apparatus for using the evaluation balloon is disclosed in U.S. Patent Publication No. 2005/0209601, entitled Multi-Stage Biomaterial Injection System For Spinal Implants, the disclosure of which is incorporated by reference.

In one embodiment, a three-dimensional mold system includes a catheter having at least one lumen and a balloon which serves as a mold form. The catheter is used to position the balloon within the nuclear cavity. A flowable, evaluation material is delivered through the catheter to the balloon. The balloon expands within the nuclear cavity and conforms to the internal geometry of the nuclear cavity. The evaluation material is then cured to form a three-dimensional mold of the nuclear cavity. The cured evaluation material is sufficiently deformable and resilient such that the three-dimensional mold can be removed from the nuclear cavity via the annulotomy through which it was inserted. In another embodiment, the flowable, evaluation material is injected directly into the nuclear cavity.

In one embodiment, the evaluation material is a low density polymer foam that is cured in situ. The cured foam is sufficiently deformable such that it permits removal of the three-dimensional mold from the nuclear cavity via the annulotomy. Upon removal, the mold is sufficiently resilient such that it returns to its cured shape.

In one embodiment, the evaluation material is a two-part reactive system that, upon mixing, cures in situ within the nuclear cavity to form the solid, three-dimensional mold. The two-part reactive system can be mixed outside or within the nuclear cavity. In one embodiment, the two-part reactive system reacts to form a foam material. In another embodiment, the two-part reactive system reacts to form a solid polymer. In yet another embodiment, the two part reactive system reacts to form a gel. In each embodiment, the solid, three-dimensional mold is sufficiently deformable that it permits removal of the three-dimensional mold from the nuclear cavity via the annulotomy. Upon removal, the mold is sufficiently resilient such that it returns to its cured shape.

According to another embodiment of the present invention, a three-dimensional mold system includes a catheter having at least one lumen and a compliant balloon that is adapted to remember an expanded configuration as defined by the inner constraints of the nuclear cavity. The compliant balloon includes a shape memory material. The shape memory properties of the balloon material are activated by the application of an external or internal stimulus to “set” the balloon material in its expanded shape. The balloon is deflated and removed from the nuclear cavity. Upon re-inflation of the balloon, the balloon returns to its “set” expanded shape as defined by the inner constraints of the nuclear cavity.

According to another embodiment of the present invention, a three-dimensional mold system includes a multi-lumen catheter and a secondary balloon disposed within a primary balloon. Each of the balloons are in fluid connection with a catheter lumen. In one embodiment, the primary balloon is made from a biocompatible material that has a high compliance allowing the primary balloon to stretch without breaking. The secondary balloon includes a shape memory material, such as for example Nitinol, another shape memory alloy, or a curable polymer material, embedded within the balloon wall. The secondary balloon is inflated within the primary balloon until both balloons substantially fill the nuclear cavity such that they conform to the features of the internal geometry of nuclear cavity. An activating mechanism is then inserted into the secondary balloon in order to activate the shape memory properties of the shape memory material embedded within the balloon walls and set the expanded shape of the secondary balloon. In another embodiment, the activating system is pre-positioned between the primary and secondary balloons.

A vacuum is optionally applied to the primary balloon to force the secondary balloon to collapse in order to permit removal of the balloons from the nuclear cavity. Upon removal, the secondary balloon is then allowed to return to its set shape as defined by the inner constraints of the nuclear cavity. According to one embodiment, the secondary balloon automatically expands to its “set” expanded shape. According to another embodiment, the secondary balloon is re-inflated until it reaches a predetermined pressure at which the secondary balloon provides a representative three-dimensional model of the nuclear cavity. The compliance of the primary balloon permits the expansion of the secondary balloon.

According to yet another embodiment of the present invention, a three-dimensional mold system includes a multi-lumen catheter and a secondary balloon disposed within a primary balloon. A shape memory mesh framework is disposed between the outer circumference of the secondary balloon and the inner circumference of the primary balloon. The secondary balloon is inflated within the primary balloon forcing the primary balloon and the shape memory mesh to conform to the internal geometry of the nuclear cavity. The shape memory properties of the shape memory mesh are then activated to set the shape of the three-dimensional mold. The inflation medium is then evacuated from the balloons. A vacuum can be applied to assist in the deflation of the balloons. The collapse of the primary balloon forces the collapse of the shape memory mesh and permits the removal of the balloons from the nuclear cavity via the annulotomy.

Upon removal, the shape memory material allows the three-dimensional mold to return to its set shape as defined by the inner constraints of the nuclear cavity. According to one embodiment, the shape memory mesh automatically expands to its “set” expanded shape. According to another embodiment, the secondary balloon is re-inflated until it reaches a predetermined pressure or temperature at which the shape memory mesh assumes its set expanded shape and provides a representative three-dimensional model of the nuclear cavity. The elasticity of the primary balloon permits the re-expansion of the shape memory mesh.

The present method is the preferred precursor procedure to implanting certain intervertebral prosthesis. In one embodiment, the intervertebral prosthesis is a mold fluidly coupled to a delivery cannula. A flowable biomaterial is delivered through a cannula into the mold located in the annulus. The delivered biomaterial is allowed to cure a sufficient amount to permit the cannula to be removed. Various implant procedures, implant molds, surgical procedures, and biomaterials related to intervertebral disc replacement suitable for use with the present invention are disclosed in U.S. Pat. No. 5,556,429 (Felt); U.S. Pat. No. 6,306,177 (Felt et al.); U.S. Pat. No. 6,248,131 (Felt et al.); U.S. Pat. No. 5,795,353 (Felt); U.S. Pat. No. 6,079,868 (Rydell); U.S. Pat. No. 6,443,988 (Felt et al.); U.S. Pat. No. 6,140,452 (Felt et al.); U.S. Pat. No. 5,888,220 (Felt et al.); U.S. Pat. No. 6,224,630 (Bao et al.), U.S. Pat. No. 7,001,431 (Felt et al.); U.S. Pat. No. 7,077,865 (Felt et al.); and U.S. Patent Publication No. 2006/0253198, entitled Multi-Lumen Mold for Intervertebral Prosthesis and Method of Using Same filed Nov. 8, 2005; 2006/0253199, entitled Lordosis Creating Nucleus Replacement Method and Apparatus filed Nov. 8, 2005 ; 2006/0265076, entitled Catheter Holder for Spinal Implant filed Nov. 8, 2005; and U.S. Ser. No. 11/420,055 entitled Mold Assembly for Intervertebral Prosthesis filed May 24, 2006, all of which are hereby incorporated by reference.

As used herein the following words and terms shall have the meanings ascribed below:

“cure” and inflections thereof, will generally refer to any chemical transformation (e.g., reacting or cross-linking), physical transformation (e.g., hardening or setting), and/or mechanical transformation (e.g., drying or evaporating) that allows the evaluation material to change or progress from a first physical state or form (generally liquid or flowable) that allows it to be delivered to the site, into a more permanent second physical state or form (generally solid) for final use in vivo. When used with regard to the method of the invention, for instance, “curable” can refer to uncured evaluation material, having the potential to be cured in vivo (as by catalysis or the application of a suitable energy source), as well as to the evaluation material in the process of curing. As further described herein, in selected embodiments the cure of a evaluation material can generally be considered to include three stages, including (a) the onset of gelation, (b) a period in which gelation occurs and the evaluation material becomes sufficiently tack-free to permit shaping, and (c) complete cure to the point where the evaluation material has been finally shaped for its intended use.

“minimally invasive mechanism” refers to a surgical mechanism, such as microsurgical, percutaneous, or endoscopic or arthroscopic surgical mechanism, that can be accomplished with minimal disruption to the entry site of the body cavity (e.g., incisions of less than about 4 cm and preferably less than about 2 cm). In some embodiments, minimally invasive mechanisms also refers to minimal disruption of the pertinent musculature, for instance, without the need for open access to the tissue injury site or through minimal skin incisions. Such surgical mechanism are typically accomplished by the use of visualization such as fiberoptic or microscopic visualization, and provide a post-operative recovery time that is substantially less than the recovery time that accompanies the corresponding open surgical approach.

“body cavity” refers to an internal three-dimensional space located within a patient's body. The space can be a natural space such as, for example, an internal region of an organ or vessel, or the space can be created via a surgical procedure, such as for example a nuclear cavity. Additionally, the space can be a natural space created through injury or abnormal development of a bodily structure.

“mold” and “balloon” will generally refer to the portion or portions of an apparatus used to receive, constrain, shape and/or retain a flowable evaluation material. A mold may include or rely upon natural tissues (such as the annular shell of an intervertebral disc or the bony shell of a vertebrae) for at least a portion of its structure, conformation or function. The mold may also be responsible to assist in removal of the three-dimensional mold from the body cavity into which it has been inserted. As such, its dimensions and other physical characteristics can be predetermined to provide an optimal combination of such properties as the ability to be delivered to a site using minimally invasive mechanism. The mold can be elastic, inelastic, porous, or non-porous.

“evaluation material” refers to a material or structure that is adapted to set to a shape generally corresponding to at least a portion of a body cavity, but be sufficiently deformable to be removed through a point of entry without damaging the entry site Examples of such evaluation materials include low density polymer foam, a balloon containing a low density polymer foam, a shaped memory material structure, a balloon containing shaped memory materials, and a balloon or other similar structure formed from a material adapted to be set by the application of an external stimulus, including heat.

“set” or “setting” refer to fixing or converting a material to substantially retain its shape when the set occurred. Examples of set or setting include curing, cross-linking, and heating.

### BRIEF DESCRIPTION OF THE SEVERAL VIEWS OF THE DRAWING

FIG. 1 is a schematic illustration of various entry paths for use in accordance with the present invention.

FIG. 2 is an exemplary catheter and mold in accordance with an embodiment of the present invention.

FIG. 3A is a schematic view of a deflated balloon positioned within a nuclear cavity in accordance with an embodiment of the present invention.

FIG. 3B is a schematic view of the balloon of FIG. 3A filled with an uncured evaluation material in accordance with an embodiment of the present invention.

FIG. 3C is a schematic view of the balloon of FIG. 3B filled with a cured evaluation material in accordance with an embodiment of the present invention.

FIG. 3D is a schematic view of the mold shown in FIG. 3C removed from the nuclear cavity in accordance with an embodiment of the present invention.

FIG. 4A-4B are schematic views of a removal mechanism provided in accordance with an embodiment of the present invention.

FIG. 5 is a schematic view of a removal mechanism provided in accordance with another embodiment of the present invention.

FIGS. 6A-6B are schematic views of a removal mechanism provided in accordance with embodiments of the present invention.

FIGS. 7A-7B are schematic views of a removal mechanism provided in accordance with yet other embodiments of the present invention.

FIG. 8 is a schematic view of a removal mechanism provided in accordance with yet another embodiment of the present invention.

FIGS. 9A-9B are schematic views of a removal assist device provided in accordance with yet other embodiments of the present invention

FIG. 10A is a schematic view of a deflated balloon positioned within a nuclear cavity in accordance with another embodiment of the present invention.

FIG. 10B is a schematic view of the balloon shown in FIG. 10A inflated with an inflation medium and positioned within the nuclear cavity in accordance with an embodiment of the present invention.

FIG. 10C. is a schematic view of an activating mechanism inserted within the inflated balloon shown in FIG. 10A in accordance with an embodiment of the present invention.

FIG. 10D is a schematic view of the balloon shown in FIG. 10A removed from the nuclear cavity in accordance with an embodiment of the present invention.

FIG. 11A is a cross-sectional schematic view of a three-dimensional mold system including a primary balloon and a secondary balloon positioned within the intervertebral disc space in accordance with an embodiment of the present invention.

FIG. 11B is a cross-sectional schematic view of an activating mechanism inserted within the secondary balloon of the three-dimensional mold system shown in FIG. 11A in accordance with an embodiment of the present invention.

FIG. 12A is a cross-sectional schematic view of a three-dimensional mold system including mesh framework disposed between a primary balloon and a secondary balloon positioned within the intervertebral disc space in accordance with another embodiment of the present invention.

FIG. 12B is a cross-sectional schematic view of the secondary balloon expanded within the primary balloon of three-dimensional mold system shown in FIG. 12A in accordance with an embodiment of the present invention.

FIG. 12C is a schematic view of the three-dimensional mold system of FIG. 12A removed from the nuclear cavity in accordance with an embodiment of the present invention.

FIG. 13A is a cross-sectional schematic view of a three-dimensional mold system including a gel material provided in accordance with an embodiment of the present invention.

FIG. 13B is a cross-sectional schematic view showing removal of a three-dimensional mold system of FIG. 13A provided in accordance with an embodiment of the present invention.

FIG. 13C is a schematic view of the three-dimensional mold system of FIG. 13A removed from the nuclear cavity in accordance with an embodiment of the present invention.

### DETAILED DESCRIPTION OF THE INVENTION

In one embodiment, the present systems and methods are directed to an improved nucletomy or total nucleus removal (TNR). Total nucleus removal refers to removal of substantially all of the nucleus from an intervertebral disc. In one embodiment, total nucleus removal is preferably removal of at least 70% of the nucleus, and more preferably at least 80% of the nucleus is removed, and most preferably at least 90% of the nucleus is removed from the intervertebral disc. TNR is the preferred precursor procedure for deploying a nucleus replacement prosthesis. The present TNR methodology, using the systems and methods according to the various embodiments of the present invention described herein, permits the nucleus replacement prosthesis to be accurately positioned within the disc space, and optimally symmetric relative to the midline of the spine.

In another embodiment, the systems and methods as described herein may be used for the evaluation of body cavities prepared for interbody fusion. With interbody fusion systems, a body cavity between the vertebrae is created. This involves removal of the nucleus, some of the annulus, and preparation of the vertebral endplates. The latter refers to the process whereby the disc and cartilage endplates are removed, exposing a portion of the endplates. To some degree a bleeding bed of vertebral endplate bone is achieved, which enhances the fusion process (allowing bone to grow from one endplate, through the interbody implant, to the other endplate). Typically during interbody fusion procedures, after removal of the disc and preparation of the endplates, the internal shape and size of the body cavity can be assessed in order to determine which size of implant is needed.

In other embodiments, the systems and methods as described herein may also be applied to kyphoplasty and vertebroplasty procedures. During a kyphoplasty procedure, a body cavity is created within the vertebral body to restore, to some extent, the natural shape of the deformed body. Typically, the vertebral body is compressed in the anterior portion, resulting in a kyphotic wedge shape. The kyphoplasty procedure seeks to distract this compressed region. Access to the vertebral body is typically obtained via a posterior or posterolateral approach, although any approach could be used. A distraction mechanism is then placed into the vertebral body, distraction is applied, and a cavity is then created once the distraction has been completed. The systems and methods as described herein can be applied to assess and evaluate the resultant cavity.

In yet other embodiments, the systems and methods as described herein may also be applied to annular repair and corpectomy procedures. In still other embodiments, the systems and methods as described herein may also be applied to non-spinal surgical procedures including meniscus repair and/or replacement, joint resurfacing, repair and/or replacement, cosmetic surgery, and drug delivery.

Although the nucleus replacement scenario is used to describe the methods and systems of this application, this is not meant to be limiting, but rather illustrative. Other cavities within the body can be assessed using these systems and methods described herein according to the various embodiments.

In one embodiment, the nucleus is divided into a plurality of regions. A preferred sequence for removing the nucleus material from each of the regions is established. The regions are preferably arranged to take into consideration the three-dimensional nature of the nucleus material. At least two different surgical instruments are typically used to remove the nucleus material from at least two of the regions. The surgical instruments are selected for optimum removal of the nucleus material from a given region. In some embodiments, different functions of multi-function surgical tools can be used to remove the nucleus material from two of the regions. In some embodiments, indicia are provided on the surgical tools to measure depth of penetration into the annulus Various sequences and surgical instruments for performing a nuclectomy are disclosed in U.S. Patent Publication No. 2006/0253199, entitled NUCLECTOMY METHOD AND APPARATUS, which is hereby incorporated by reference.

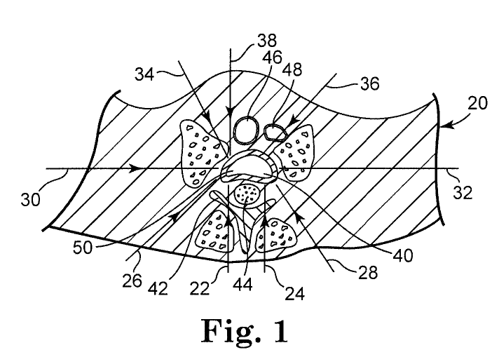

FIG. 1 is a cross-sectional view of a human body 20 showing various access paths 22 through 38 to the intervertebral disc space 40 for performing the method of the present invention. Intervertebral disc space refers generally to the space between adjacent vertebrae. The posterior paths 22, 24 extend either between superior and inferior transverse processes 42, or between the laminae (interlaminar path) on either side of the spinal cord 44. The posterolateral paths 26, 28 are also on opposite sides of the spinal cord 44 but at an angle of about 35-45 degrees relative to horizontal relative to the posterior paths 22, 24. The lateral paths 30, 32 extend through the side of the body. The anterior path 38 and anterolateral path 34 extend past the aorta iliac artery 46, while the anterolateral path 34 is offset from the inferior vena cava, iliac veins 48.

Depending on the disc level being operated on, and the patient anatomy, generally, the aorta and vena cava split at the L4 vertebral body. At L5S1 the approach is typically a midline anterior approach. At L4/5 the approach may be either midline anterior or anterolateral, depending on the patient anatomy and how easy it is to retract the vessels. In some usages, the anterior approach is deemed a midline approach and the anterolateral approach is deemed an angled approach offset from the midline anterior approach.

The present method and apparatus use one or more of the access paths 22 through 38. While certain of the access paths 22 through 38 may be preferred depending on a number of factors, such as the nature of the procedure, any of the access paths can be used with the present invention.

In one embodiment, delivery catheter instruments are positioned along two or more of the access paths 22 through 38 to facilitate preparation of the intervertebral disc space 40. Preparation includes, for example, formation of two or more annulotomies through the annular wall, removal of some or all of the nucleus pulposus to form a nuclear cavity 50, imaging of the annulus and/or the nuclear cavity 50, and positioning of the present three-dimensional mold in the nuclear cavity. In another embodiment, the present three-dimensional mold is positioned in the nuclear cavity 50 without use of delivery catheters.

In one embodiment, an evaluation mold is positioned in the nuclear cavity and a fluid is delivered to the evaluation mold so that the mold substantially fills the nuclear cavity. The evaluation mold is used to estimate the quantity of nucleus material removed as well as the position of the mold within the nuclear cavity. The evaluation mold can also be used to estimate the geometry of the nuclectomy. A method and apparatus for using the evaluation mold is disclosed in U.S. Patent Publication No. 2005/0209601, entitled Multi-Stage Biomaterial Injection System For Spinal Implants, the disclosure of which is incorporated by reference.

Imaging techniques known to those of skill in the art can be used to estimate the volume, three-dimensional geometry, and position of the evaluation mold and/or the three-dimensional mold in the nuclear cavity. Exemplary imaging techniques include arthroscopic and fluoroscopic imaging. The visual estimations of the volume and geometry of the evaluation mold can be compared to the volume and geometry of the three-dimensional mold that is formed in accordance with various embodiments of the present invention. This further aids the surgeon performing the procedure in determining whether additional removal of the nucleus is required. Imaging techniques can be used throughout the entire procedure of forming a three-dimensional mold system. The balloon and or evaluation material can be radiopaque. A radiopaque mold can assist in imaging the mold in the cavity and can eliminate the need for additional imaging equipment.

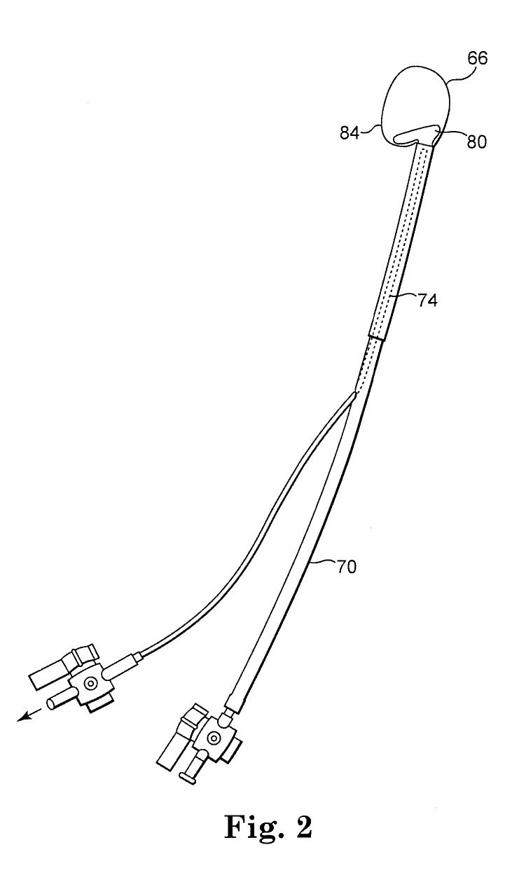

FIG. 2 illustrates one embodiment of a system 60 for measuring the shape of the nuclear cavity 50 including a three-dimensional mold 66 in accordance with the present invention. The three-dimensional mold system 60 is similar to the biomaterial injection system shown and described in U.S. Patent Publication No. 2006/0253198, entitled Multi-Lumen Mold for Intervertebral Prosthesis and Method of Using Same, which is hereby incorporated by reference. The three-dimensional mold system 60 includes a catheter 70 having one or more lumens 74 adapted for delivering one or more evaluation materials 80 to a balloon 84 which serves as the three-dimensional mold form. The balloon 84 can be a single portal or a bi-portal balloon.

The balloon 84 is made from a biocompatible material that has a high compliance allowing the balloon 84 to stretch without breaking. More particularly, the compliance of the balloon 84 facilitates conformal expansion of the balloon within the nuclear cavity at inflation pressures ranging from about 20 psi to about 200 psi. Additionally, the balloon 84 should exhibit a high tensile strength and a high tear resistance. The balloon material may or may not adhere to the evaluation material. Exemplary balloon materials include, but are not limited to, the following: silicone elastomers, soft grades of polyurethane, latex rubber, nitrile rubber, polyvinylchloride, and polyethylene terephthalate, and other similar materials exhibiting the desired physical properties. According to a further embodiment, the balloon 84 can include a shape memory material.

Additionally, a lubricant or other surface modifying agent may be applied to the outer surface of the balloon 84 to enhance the lubricity of the balloon. Enhanced lubricity of the balloon outer surface aids in removal of the balloon 84 and/or mold, reducing the risk of damage to the annulus and surrounding tissue. The enhanced lubricity of the balloon 84 may be achieved through chemical modification of the outer surface of the balloon 84. Alternatively, the lubricant may be introduced directly into the nuclear cavity 50 prior to insertion of the balloon 84.

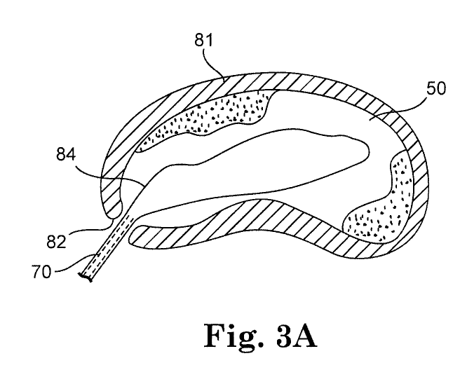

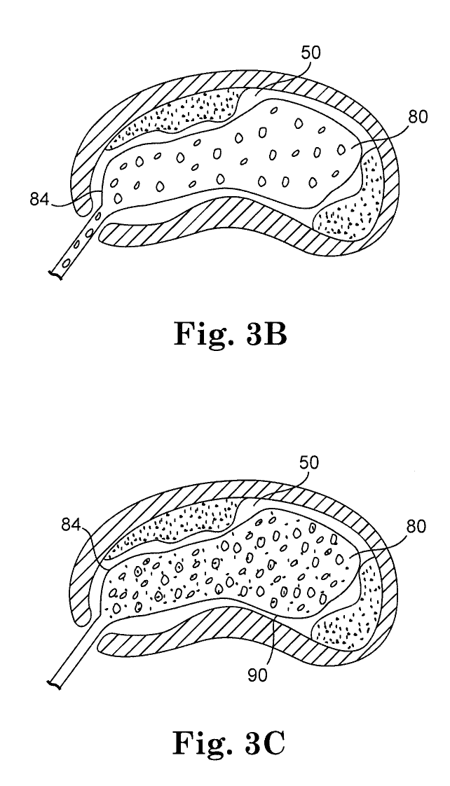

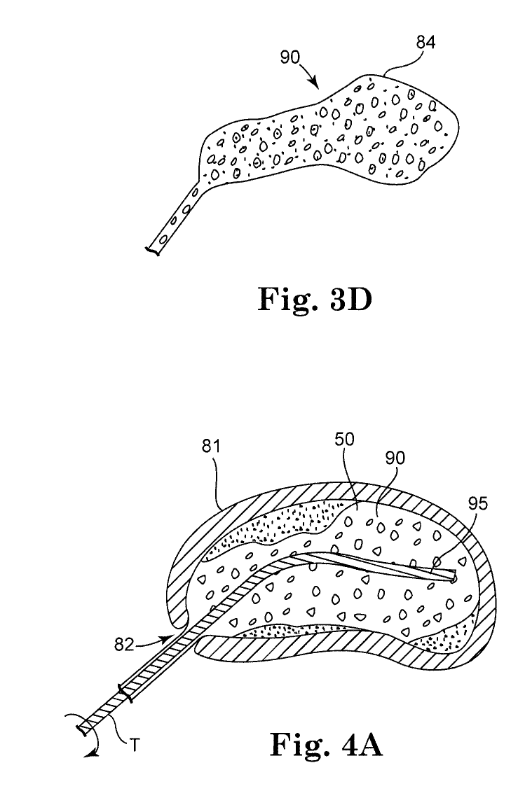

FIGS. 3A-3D are schematic views of the three-dimensional mold system 60 in use according to one embodiment of the present invention. In use, the deflated balloon 84 is dimensioned to locate in the nuclear cavity 50 through annulotomy 82 formed in the annulus 81, as shown in FIG. 3A. The balloon 84 is then filled with an uncured evaluation material 80 that can expand the balloon 84, and then when cured conforms to the interior of the nuclear cavity 50, as shown in FIG. 3B. As the evaluation material 80 is delivered to the balloon 84, the balloon 84 expands to substantially fill the nuclear cavity 50. Contrast media, such as radiopaque dye or another contrast solution, may also be delivered to the balloon 84 such that the filling of the nuclear cavity 50 can be visualized by the surgeon performing the procedure. When cured, the evaluation material 80 conforms to the nuclear cavity 50 such that it conforms to the internal geometry and surface features of the nuclear cavity and forms a solid, three-dimensional mold 90 of the nuclear cavity 50, as shown in FIG. 3C. Upon removal from the nuclear cavity 50, the three-dimensional mold 90 is able to return to its cured shape, as shown in FIG. 3D.

Once removed from the nuclear cavity, the three-dimensional mold 90 can be evaluated quantitatively and qualitatively. For example, the mold 90 can be computer scanned to create a computer model of the mold 90, and hence, a computer image approximating the internal geometry of the nuclear cavity 50.

According to another embodiment, a balloon 84 is not required. The evaluation material may be delivered directly into the nuclear cavity 50 via injection. The evaluation material 80 can then be cured within the nuclear cavity 50 to form a three-dimensional mold. The three-dimensional mold 90 is illustrated in FIG. 3D.

According to one embodiment of the present invention, the three-dimensional mold 90 should have the ability to reversibly deform. Additionally, the three-dimensional mold 90 should exhibit high compliance, high tear resistance, and low residual deformation. According to a further embodiment of the present invention, the mold 90 should be able to compress to about 10% to about 40% of its fully deployed size and then re-expand about 90% to about 100% of its fully deployed size upon removal from the nuclear cavity.

The three-dimensional mold 90, according to various embodiments of the present invention, may be formed from a wide variety of biocompatible materials, including elastomers, polyurethanes, elastomeric polyolefins and polyolefin blends, silicone rubbers, silicone based polymer systems, gels including hydrogels, or composites thereof. Exemplary elastomers include copolymers of silicone and polyurethane, polyolefins, such as polyisobutylene and polyisoprene, neoprene, nitrile, vulcanized rubber and combinations thereof. Suitable hydrogels include natural hydrogels, and those formed from polyvinyl alcohol, acrylamides such as polyacrylic acid and poly (acrylonitrile-acrylic acid), polyurethanes, polyethylene glycol, poly (N-vinyl-2-pyrrolidone), acrylates such as poly (2-hydroxy ethyl methacrylate) and copolymers of acrylates with N-vinyl pyrrolidone, N-vinyl lactams, acrylamide, polyurethanes and polyacrylonitrile. The hydrogel materials may further be cross-linked to provide further strength to the implant. Other useful gel and gel foamed materials for forming a three-dimensional mold 90 are described in U.S. Pat. Nos. 7,222,380 and 7,226,484, both of which are herein incorporated by reference in their entirety. In certain instances a foaming agent may be added to produce a foam of one or more of the materials provided above.

In a further embodiment, the evaluation material 80 may be a two-part reactive system that upon mixing cures in situ within the nuclear cavity 50 to form the solid, three-dimensional mold 90. The two-part reactive system can be mixed inside or outside the nuclear cavity. Whatever the materials used to form the two-part system, the resultant three-dimensional mold 90 should be sufficiently deformable such that it can be removed from the nuclear cavity 50 through the annulotomy 82 without damaging the annulus 81. When cured, the evaluation material 80 is able to conform to the internal geometry of the nuclear cavity 50 and annulus 81 and retain its adopted shape upon removal.

According to another further embodiment of the present invention, the evaluation material 80 may be a reactive two part system forming a soft resilient foam upon mixing. A first flowable material forming the first part (Part A) of the two part reactive system is delivered through a first catheter lumen and into the balloon 84. The first flowable material fills the balloon 84 such that the balloon 84 substantially fills the nuclear cavity 50. A second flowable material (Part B) is delivered through a second catheter lumen and into the balloon 84. Part A reacts with Part B upon contact to form the three-dimensional mold 90. The three-dimensional mold 90 is constrained by and conforms to the internal geometry of the nuclear cavity 50. In one embodiment, the cured material is sufficiently deformable such that it permits removal of the three-dimensional mold from the nuclear cavity 50 via the annulotomy 82. The foam mold is sufficiently resilient such that it returns to its cured shape outside of the nuclear cavity. In one embodiment, the evaluation material 80 is cured using one or more of heat, ultraviolet radiation, visible light, or radio frequency energy.

According to a further embodiment, the evaluation material may be a reactive two part system forming a highly stretchable polymer upon mixing and curing in situ.

According to another further embodiment, the evaluation material may be a reactive two part system containing a significant amount of an inert solvent (plasticizer) to form a gel.

According to yet another embodiment of the present invention, the evaluation material includes a biocompatible polymer having a thermal transition between about 35° C. and about 75° C. The evaluation material is injectable above the thermal transition temperature. Below the thermal transition temperature the evaluation material is solid, pliable, and is able to retain the shape of the nuclear cavity. According to one embodiment, the evaluation material is heated above the thermal transition temperature and then injected to fill the balloon. Once inside the body the evaluation material cools to body temperature below the thermal transition temperature to form the three-dimensional mold.

Whatever the selected material or materials used to form the mold 90, the mold 90 should exhibit high compliance, a high tensile strength, and a high tear resistance. According to one embodiment, the mold 90 should exhibit a Young's modulus that allows the mold 90 to be deformed up to about 200% to about 500% of its original shape with out tearing or breaking in response to a tensile forces ranging from about 0.1 MPa to about 10 MPa are placed upon it, and then return to its original shape once the force is removed.

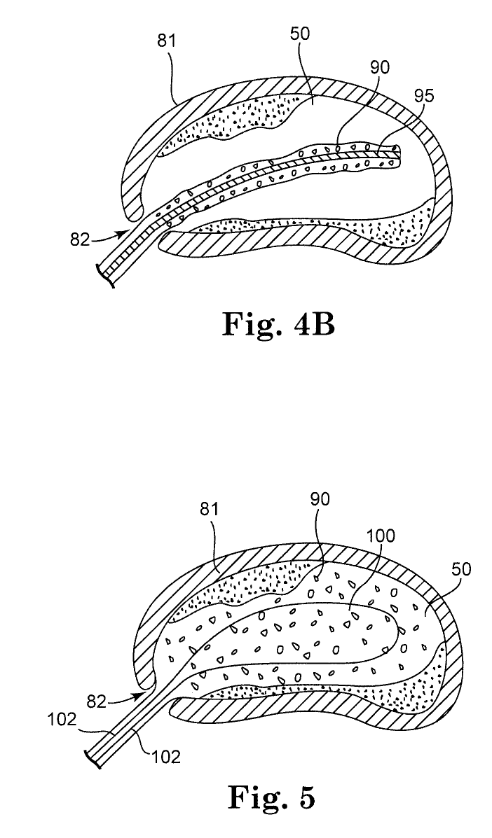

The system 60 may be provided with one or more removal mechanisms according to various embodiments of the present invention, as illustrated in FIGS. 4A-9B, to facilitate the removal of the three-dimensional mold 90.

According to one embodiment, shown in FIGS. 4A and 4B, the removal mechanism can be a flexible, threaded member 95. According to one embodiment, the flexible, threaded member 95 can be a wire. The flexible, threaded member 95 is inserted within the nuclear cavity 50 prior to curing of the evaluation material 80. According to a further embodiment of the present invention, if balloon 84 is used as the mold form, the flexible threaded member 95 may be attached to a distal inner surface of the balloon 84. This arrangement also facilitates insertion of the balloon 84 into the annulus by applying a longitudinal force to stretch the balloon 84 so that it can be easily inserted through the annulotomy 82 and positioned within the nuclear cavity 50.

Upon curing, the mold 90 forms around the flexible threaded member 95. To facilitate removal of the mold 90, a torque T is applied to the threaded member 95 by rotating or twisting the threaded member 95 in a clockwise or counter clockwise direction as determined by the inner geometry of the nuclear cavity and/or the surgeon's preference. As shown in FIG. 4B, rotation of the threaded member 95 causes the mold 90 to compress and wrap or twist about the flexible member 95 such that it can be more easily removed through the annulotomy 82. The protrusions provide additional surface area around which the mold 90 can wrap or twist. Once outside the patient's body, the rotational forces T applied to the threaded member 95 can be released, and the mold 90 can return to its adopted shape.

According to another embodiment, as illustrated in FIG. 5, the removal mechanism can be a looped member 100. According to one embodiment, the looped member 100 is a wire loop. According to another embodiment, the looped member 100 maybe made from a polymeric or other biocompatible material capable of adopting a looped configuration. The looped member 100 may include one or more tethers 102 which facilitate its removal from the nuclear cavity 50. The looped member 100 is compressed and delivered via a catheter or other delivery member through the annulotomy 82 and into the nuclear cavity 50. The looped member 100 may be delivered before or after the delivery of the evaluation material 80. After the looped member 100 has been properly positioned within the nuclear cavity, the evaluation material 80 can be cured around the looped member 100 such that it creates a three-dimensional mold 90 of the inner geometry of the nuclear cavity 50. Placing tension on the tethers 102 causes the looped member to pull on the surrounding evaluation material such that the mold 90 becomes compresses about the looped member, facilitating removal of the mold 90 from the nuclear cavity. Once tension has been released off of the tether 102, the mold 90 returns to its adopted shape.

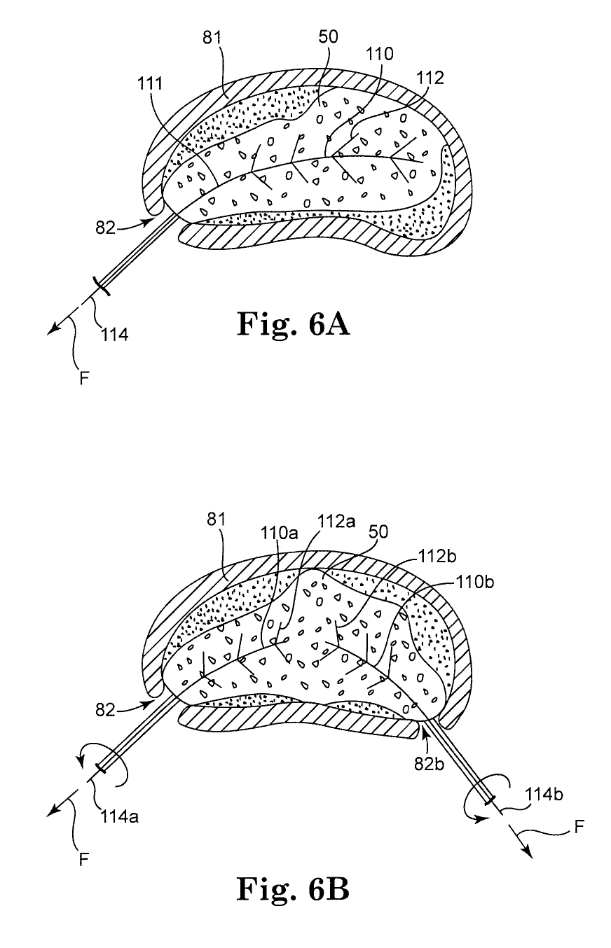

According to yet another embodiment, as shown in FIGS. 6A and 6B, the removal mechanism 111 includes at least one elongated wire 110 and a plurality of expandable tines or fins 112. FIG. 6A shows a single finned elongated wire 110 inserted through the annulotomy 82 into the nuclear cavity 50. FIG. 6B shows two finned elongated wires 10a and 10b. Referring to FIG. 6B, the first finned elongated wire 10a is inserted through a first annulotomy 82a and the second finned elongated wire 110b is inserted through a second annulotomy 82b. As shown in FIGS. 6A and 6B, the fins 112 have assumed an expanded configuration within the nuclear cavity 50. The mold 90 is cured about the finned elongated wires 110.

According to one embodiment, as shown in FIG. 6A, to remove the mold 90, a longitudinal force F is placed on the distal end 114 of the removal mechanism 111 in a proximal direction. The longitudinal force F causes the fins 112 to collapse, compressing the mold 90 such that it facilitates the removal of the mold 90 from the nuclear cavity 50 via the annulotomy 82. When the force F is removed, the fins 112 expand and the mold 90 returns to its adopted shape.

According to another embodiment, as shown in FIG. 6B, to remove the mold 90, the surgeon grips the distal ends 119A, 119B of each wire 10a, 110b and twists or rotates each of the wires in a clockwise or counter clockwise direction C. The mold 90 is then removed via either annulotomy 82a or 82b. Once the rotation forces C about the wire have been released, the mold 90 returns to its adopted shape

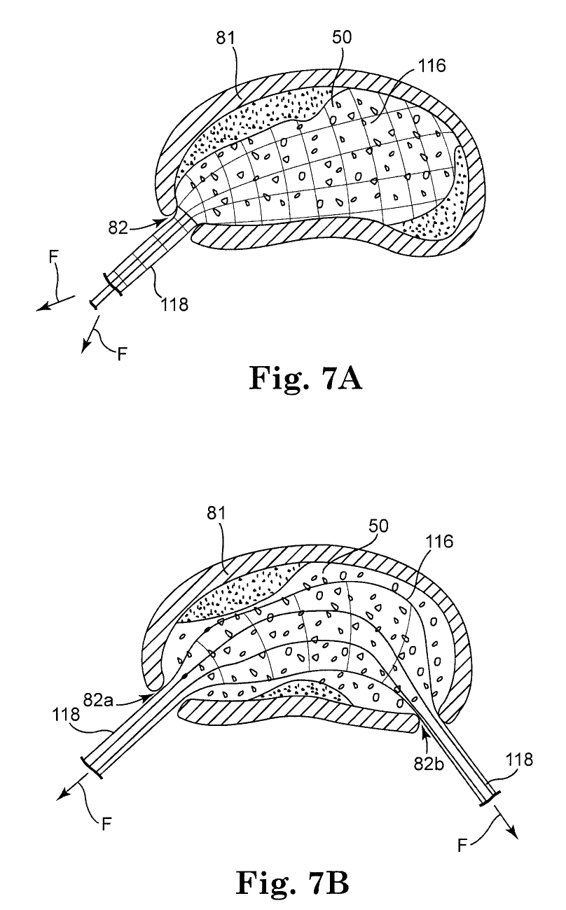

According to yet another embodiment as shown in FIGS. 7A and 7B, the removal mechanism can include a flexible, mesh compression member 116. The flexible mesh compression member 116 can be used in applications where there is a single annulotomy 82, as shown in FIG. 7A, or dual annulotomies 82a and 82b, as shown in FIG. 7B.

According to one embodiment, the flexible mesh compression member 116 includes a mesh frame work having at least one tension member 118 accessible to the surgeon. The compression material 116 forming the framework may allow for some seepage of the evaluation material 80 through its pores. The compression member 116 is inserted into the nuclear cavity 50 prior to the delivery of the evaluation material 80. The evaluation material 80 is delivered to the nuclear cavity 50 within the compression member 116 substantially filling the nuclear cavity 50. The evaluation material is then cured such that a mold 90 of the cavity 50 is created.

To remove the mold 90, tension is applied to the tension member 118 by placing a force on the tension member 118 in a proximal direction causing the compression member 116 to compact and compress the mold 90, facilitating its removal from the nuclear cavity 50. As shown in FIG. 7B the compression member 116 can be compressed by placing proximal forces F in opposite directions on each on of the tension members 118. The compressed mold 90 then can be removed via one of the annulotomies 82a or 82b at the discretion of the surgeon.

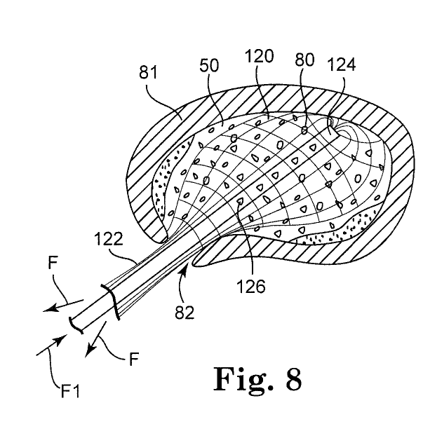

According to a further embodiment, as shown in FIG. 8 a removal mechanism includes a compression member 124 and a plurality of tension members 120. In the illustrated embodiment, the evaluation material 80 is delivered through the compression member 124. The tension member 120 is preferably a mesh material having at least one member 122 accessible by the surgeon. In the illustrated embodiment, the mesh member 120 is preferably attached to distal end of the compression member 124. Alternatively, the compression member 124 is inserted into the tension member 120 located in the nuclear cavity 50.

The compression member 124 preferably includes a plurality of apertures 126 through which the evaluation material 80 is delivered. Once the tension member 120 and the compression member 124 are positioned in the nuclear cavity 50, the evaluation material 80 is delivered via the compression member 124. The evaluation material expands the mesh of the tension member 120 to substantially fill the nuclear cavity 50.

The evaluation material 80 is then cured to form the mold 90. To remove the mold 90, force F is applied to the members 122 by the surgeon in a proximal direction 121. Simultaneously, opposing force F1 is applied to the compression member 124 to resist the force F. The combination of forces F and F1 cause the evaluation material 80 to compress, permitting removal from the nuclear cavity 50 through the annulotomy 82. Once the forces F and F1 are released, the mold 90 returns to its adopted shape.

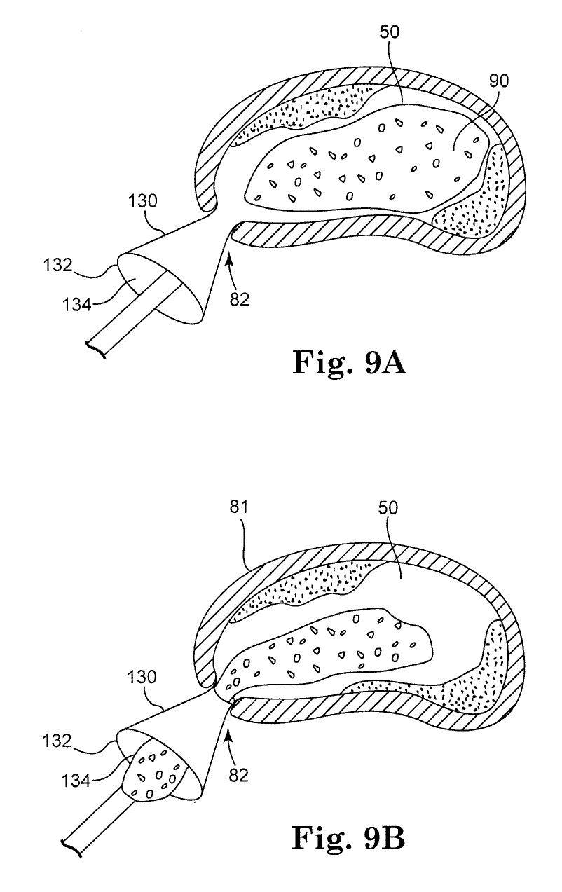

According to a further embodiment, a removal assist device 130, shown in FIGS. 9A and 9B, can be provided to assist with the removal of the three-dimensional mold 90 from the nuclear cavity 50. FIGS. 9A and 9B show the removal assist device 130 in place during removal of a representative embodiment of the three-dimensional mold 90. The removal assist device 130 protects the annulus and surrounding tissue from further damage due to removal of the three-dimensional mold 90. According to one embodiment, the removal assist device 130 is flared at a proximal end 132 to allow for re-expansion of the mold 90 as it exits the annulotomy 82 and is no longer subject to any form of constraint. According to another embodiment, the removal assist device 130 need not be flared, but may have a cannula-like configuration. According to a further embodiment, the inner lumen 134 of the removal assist device 130 may include a lubricant or other suitable surface treatment to ease removal of the mold 90. The removal assist device 130 may be used in combination with one or more various embodiments of the removal mechanisms as described above with reference to FIGS. 4A-8.

According to yet another embodiment, the mold 90 also can be removed using one or more surgical instruments adapted for removal and retrieval of the three-dimensional mold. According to various embodiments of the present invention, the mold 90 may be removed using any one of a blunt edge trephine, graspers, hemostat, pliers, hooks, etc.

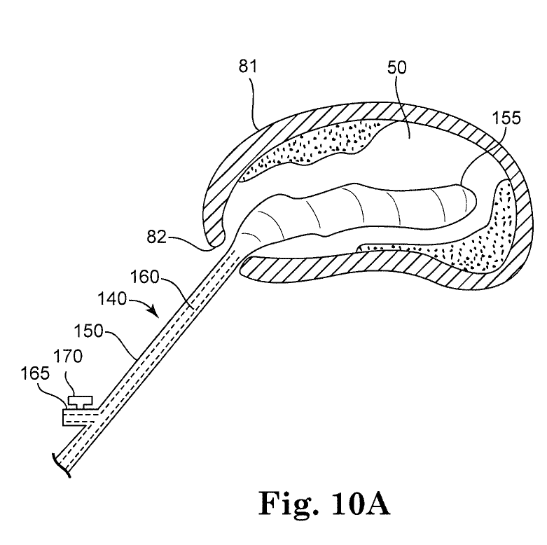

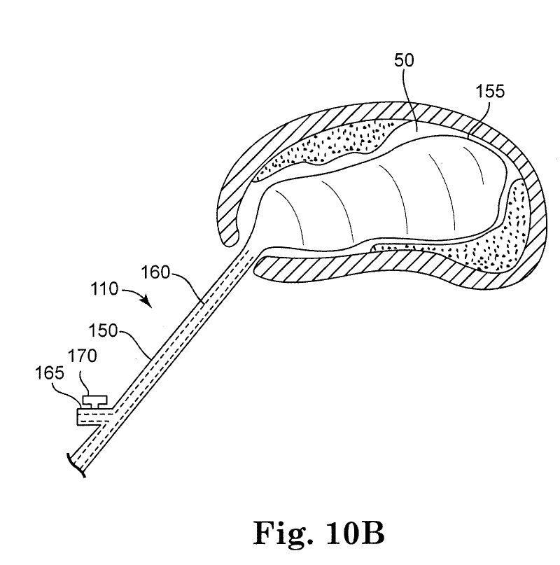

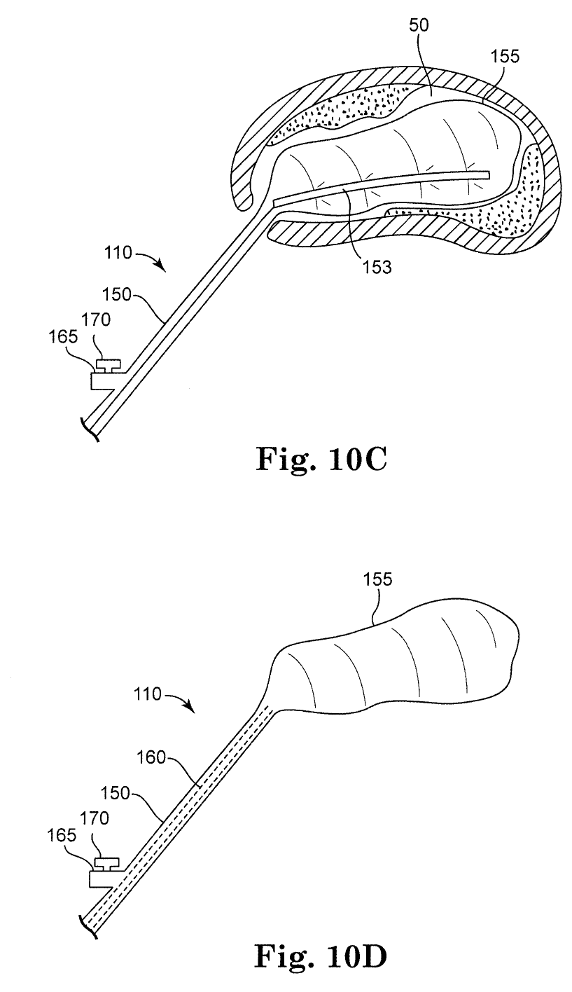

FIGS. 10A-10D are schematic views of a system 140 for modeling the internal geometry of a nuclear cavity 50 for use in accordance with another embodiment of the present invention. The system 140 includes a catheter 150 and a compliant balloon 155 that is adapted to remember an expanded configuration as defined by the inner constraints of the nuclear cavity 50.

FIG. 10A shows the balloon 155 in a deflated state. The balloon 155 is filled with an inflation medium, as shown in FIG. 10B. The inflation medium is typically a gas or a liquid. As the inflation medium is delivered to the balloon 155, such that the balloon 155 expands to substantially fill the nuclear cavity 50. According to an optional embodiment, as shown in FIG. 10C, activating mechanism 153 is inserted within the balloon, to activate the balloon's memory properties such that it remembers its expanded configuration within the constraint of the nuclear cavity 50. The activating mechanism 153 can be for example a heat source, a heat sink, an electromagnetic radiation generator, such as for example infrared or ultraviolet, magnetic field generator, etc. In another embodiment, the inflation medium is the activating mechanism, such as for example by causing a chemical reaction with the balloon 155. Upon removal from the nuclear cavity 50, the balloon 155 is returned to an expanded configuration as defined by the inner constraints of the nuclear cavity, as shown in FIG. 10D.

The catheter 150 can include one or more lumens 160 in fluid connection with the balloon 155. At least one lumen 160 is configured to deliver an inflation medium to the balloon 155. For example, the inflation medium can be a sterile saline solution or a gel. Alternatively, the inflation medium can be carbon dioxide, air or other gas. Contrast media, such as radiopaque dye or another contrast solution, may also be delivered to the balloon 155 such that the filling of the nuclear cavity 50 can be visualized by the surgeon performing the procedure.

The inflation media is preferably delivered through the catheter lumen 160 to the balloon 155 via an injection manifold 165 accessible to the surgeon external of the patient's body. The injection manifold 165 includes one or more appropriate mechanisms 170 (e.g. sealing valves) for maintaining sufficient pressure in the balloon 155 to allow the balloon 155 to fill the nuclear cavity 50 and conform to the internal geometry of the nuclear cavity 50 such that a representative mold of the nuclear cavity 50 can be obtained.

In one embodiment, the catheter 150 is configured to allow for the continuous delivery of inflation medium to the balloon 155 in order to maintain a constant pressure in the balloon 155. The pressure required to inflate the balloon 155 and fill the nuclear cavity 50 such that the balloon 155 conforms to the internal geometry of the nuclear cavity 155 can be referred to as the predetermined pressure. Later, when the balloon 155 is deflated and removed from the nuclear cavity, the balloon 155 can be re-inflated to the predetermined pressure in order to give a representative three-dimensional model of the internal geometry of the nuclear cavity 50. Alternatively, the pressure can be combined with an activating mechanism, such as heated gas, to cause the balloon 155 to fill the nuclear cavity 50, and take a set shape conforming to the internal geometry of the cavity. When the balloon 155 is deflated and removed, it can be re-inflated to the predetermined pressure, or less, as appropriate to give a representative three-dimensional model.

In one embodiment, the balloon 155 includes a shape memory material or a material that takes a set when exposed to an external stimuli. Shape memory materials are materials that have one or more properties that are altered in a controlled fashion by the application of an external stimuli, such as stress, temperature, moisture, pH, electric, or magnetic fields. For example, shape memory polymers are polymer materials which may be returned from a deformed state to their original shape or “permanent configuration” via an external stimulus. The external stimulus is usually temperature, as in the case of thermally activated shape memory polymers, but can also be the application of an electric or magnetic field, light, or a change in pH. Magnetic shape memory alloys are materials that change their shape in response to a significant change in the magnetic field.

According to one embodiment, the balloon 155 includes a shape memory polymer. Upon inflation and expansion of the balloon 155 within the constraint of internal cavity 50, the polymer chains become permanently stretched in areas where substantially all of the nuclear material has been removed and remain un-stretched over areas where removal of the nucleus material is still desirable. The balloon 155 is deflated and withdrawn from the nuclear cavity 50. The balloon 155 can then be re-inflated to the same approximate pressure that was reached during inflation within the nuclear cavity. The stretching of the polymer chains allows the balloon 155 to “remember” its expanded shape within the constrains within the nuclear cavity 50. Exemplary polymers include, but are not limited to, the following: polyurethanes, polyethylene terephthalate, polyvinylchloride, silicone elastomers, and other suitable polymers.

FIG. 10C is schematic view of an embodiment in accordance with the present invention in which the balloon 155 includes a shape memory material in which the shape memory properties of the material are triggered by the application of an external stimulus. The shape memory properties of the balloon material can be triggered by exposure of the balloon 155 to an external stimulus such as UV light, radio frequency, radiation, magnetic field, and temperature. As shown in FIG. 10C, an activation probe or mechanism 153 is delivered to the balloon 155 via one of the catheter lumens. The external stimulus is delivered to activate the shape memory properties of the balloon material. The balloon is then deflated and removed from the nuclear cavity. The balloon is then re-inflated outside of the patient's body and returns to expanded shape as defined by the constraints of the nuclear cavity, as shown in FIG. 10D. In some embodiments, a second application of the external stimulus may be necessary to prompt the balloon to return to its “remembered” shape.

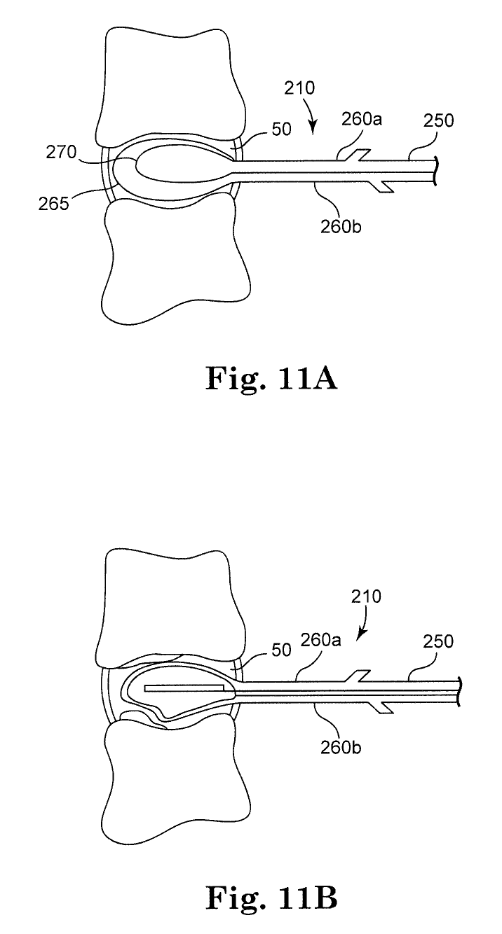

FIGS. 11A-11B are schematic views of a system 210 for modeling the internal geometry of a nuclear cavity 50 in use in accordance with another embodiment of the present invention. The system includes a catheter 250 having at least two lumens 260a, 260b and primary and secondary balloons 265, 270. The secondary balloon 270 is disposed within the primary balloon 265.

The primary balloon 265 is made from a biocompatible material having a high compliance facilitating the balloon 265 to stretch without breaking. Additionally the balloon 265 should exhibit a high tensile strength and a high tear resistance. Exemplary balloon materials include, but are not limited to, the following: silicone elastomers, soft grades of polyurethane, latex rubber, nitrile rubber, and other similar materials exhibiting the desired properties.

In one embodiment, the secondary balloon 270 includes a shape memory material, such as Nitinol or another shape memory alloy, embedded within the balloon wall. The secondary balloon 270 is inflated within the primary balloon 265 until both balloons substantially fill the nuclear cavity 50 such that they conform to the features of the internal geometry of nuclear cavity 50. An activating mechanism is then inserted into the secondary balloon in order to activate the shape memory properties of the shape memory alloy embedded within the balloon walls. According to one embodiment of the present invention, the temperature of the inflation media (either heat or cold), such as water or contrast solution, is used to activate the shape memory alloy.

Once in the expanded configuration, as defined by the inner constraint of the nuclear cavity 50, of the secondary balloon is set, the inflation medium is evacuated from the balloons. The secondary balloon 270 can be further collapsed by the application of a vacuum to the outer, primary balloon. The force of the primary balloon collapsing about the secondary balloon, causes the collapse of the secondary balloon, such that it can be removed through the annulotomy through which the balloons were inserted. Once outside the constraints of the nuclear cavity 50, the secondary balloon automatically expands to its set shape to give a three-dimensional model or mold of the nuclear cavity. The compliance of the primary balloon 265 permits the expansion of the secondary balloon 270.

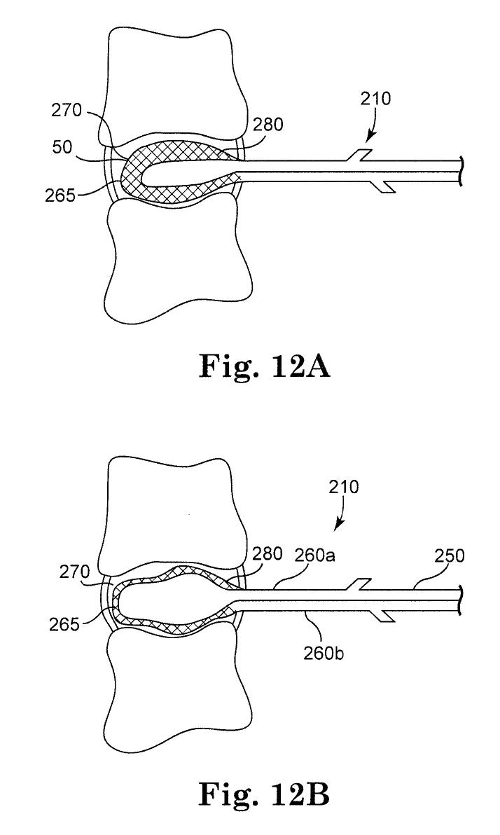

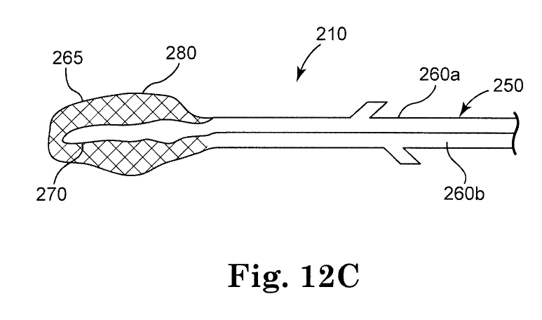

FIGS. 12A-12C are schematic views of the system 210 for modeling the internal geometry of a nuclear cavity 50 in use in accordance with another embodiment of the present invention. In one embodiment in accordance with the present invention, a shape memory mesh 280 or other framework is disposed between the inner circumference of the primary balloon 265 and the outer circumference of the secondary balloon 270. The secondary balloon 270 is inflated with an inflation medium within the primary balloon 265 until both balloons substantially fill the nuclear cavity 50. The expansion of the secondary balloon 270 within the primary balloon 265 forces the mesh framework 280 to expand and conform to the internal geometry of the nuclear cavity 50. An activating mechanism can optionally be inserted into the secondary balloon in order to activate the shape memory properties of the mesh 280 (see e.g., FIG. 10C. According to one embodiment of the present invention, the temperature of the inflation media (either heat or cold), such as water or contrast solution, is used to activate the shape memory alloy.

Once the expanded configuration of the mesh 280, as defined by the inner constraint of the nuclear cavity 50, is set, the inflation medium is evacuated from the balloons. The mesh framework 280 can be collapsed by the application of a vacuum to the primary balloon 265. The force of the primary balloon 265 collapsing about the mesh framework 280 causes the mesh framework to also collapse such that mesh framework 280 can be removed through the annulotomy through which the balloons were inserted. Once outside the constraints of the nuclear cavity 50, the mesh framework 280 automatically expands to substantially the set shape to give a three-dimensional model or mold of the nuclear cavity 50. The elasticity of the primary balloon 265 permits the expansion of the mesh framework 280. The secondary balloon 270 need not be inflated for the mesh framework 280 to return to its set expanded configuration as defined by the constraints of the nuclear cavity 50. Alternatively, the second balloon 270 can be re-inflated to a predetermined pressure in order for the mesh 280 to return to its set expanded shape as defined by the inner constraints of the nuclear cavity 50.

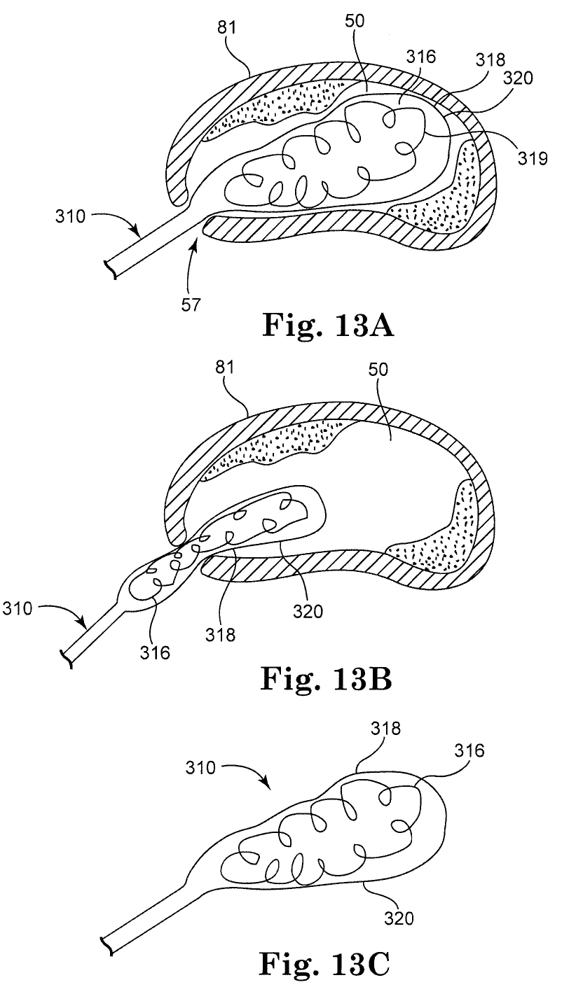

According to yet another embodiment of the present invention, as shown in FIGS. 13A- 13C, a three-dimensional mold system 310 can be filled with a shape memory gel or gel-polymer composite to form a mold 318. The gel material 316 is optionally impregnated with elongated reinforcing fibers 319 that increase the tensile strength of the resulting mold 318. According to this embodiment, a balloon 320 is filled with the gel or gel polymer composite material 316 that, when cured, conforms to the interior of the nuclear cavity 50, as shown in FIG. 13A, to form the mold 318. During removal from the nuclear cavity 50, as shown in FIG. 13B, the gel material 316 allows the mold 318 to deform so as to facilitate removal of the mold 318 from the nuclear cavity 50 via the annulotomy 82.

Upon removal from the nuclear cavity 50, the three-dimensional mold 318 is able to return to its cured shape, as shown in FIG. 13C. Exemplary shape memory gel forming materials are described in U.S. Pat. Nos. 7,222,380 and 7,226,484, which are herein incorporated by reference. The gels, as described in the '380 and '484 patents, exhibit high tear resistance, and high tensile strength. Additionally, articles formed form these gels can exhibit shape memory properties. That is, they are capable of returning to their original shape after significant deformation. According to a further exemplary embodiment, the gel and gel composites may be foamed.

In an alternate embodiment, the gel material 316 is introduced directly into the nuclear cavity 50 without the balloon 320.

### Nuclectomy Procedure

An illustration of the surgical use of one embodiment of the nuclectomy procedure including the formation of a three-dimensional mold of the internal geometry of the nuclear cavity is outlined below:
1) A nuclectomy is performed by surgically accessing the nucleus through one or more annulotomies and removing at least a portion of the nucleus of the disc to form a cavity. Preferably substantially all of the nucleus is removed from the disc. The cavity is preferably symmetric relative to the spine.
2) The distal (patient end) portion of a device of this invention is inserted into the surgical site and intervertebral space. In one embodiment, the distal tip contains a deflated mold. The mold is then inserted into the intervertebral disc space by pushing the distal end of the biomaterial delivery portion in a longitudinal direction through the annulotomy in the direction of the disc to the extent necessary to position the mold only into the nuclear cavity.
3) Imaging techniques, such as arthroscopic or fluoroscopic imaging, can be used throughout the procedure and may be used to further evaluate the nuclear cavity before and after insertion of the mold. Further, imaging can be used to monitor the formation of the mold in situ, and later to evaluate the volume, position, and three-dimensional geometry of the mold. The volume and geometry of the three-dimensional mold can be compared to that obtained from an evaluation mold, if used, prior to the insertion and formation of the three-dimensional mold.
4) Optionally, if pre-distraction of the intervertebral disc is needed when the patient has pre-existing disc height loss, it can be accomplished using any suitable intervertebral distraction mechanism, including both external and internal mechanism. Internal distraction can be accomplished by using an apparatus similar to that of the invention, e.g., by first delivering a suitable fluid (e.g., saline or contrast solution) into the mold in order to exert a force sufficient to “distract” the intervertebral joint to the desired extent. After the distraction, the solution can be removed from the mold by applying vacuum. It is optional either to use the same mold for hosting the injectable biomaterial or to replace the distraction mold with a new mold.
5) The components of a biomaterial delivery system are assembled as generally illustrated in FIG. 2.
6) The balloon is expanded within the nuclear cavity such that it fills the nuclear cavity and conforms to the internal geometry of the nuclear cavity. Pressure is maintained within the balloon during formation of the three-dimensional mold.
7) The three-dimensional mold is removed from the nuclear cavity via the annulotomy through which it was delivered.
8) Upon removal, the three-dimensional mold is expanded to its three-dimensional shape as defined by the inner constraints of the nuclear cavity.
9) The volume, geometry, and symmetry of the three-dimensional mold is evaluated and compared to images taken of the nuclear cavity before and during the procedure. Optionally, the volume, geometry, and symmetry of the three-dimensional mold is evaluated and compared to images of an evaluation mold of the nuclear cavity prior to formation of the three-dimensional mold.
10) Optionally, the removal of the nucleus can be repeated as necessary until the nuclear cavity achieves a desired internal geometry.

Patents and patent applications disclosed herein, including those cited in the Background of the Invention, are hereby incorporated by reference. Other embodiments of the invention are possible. Although the description above contains many specificities, these should not be construed as limiting the scope of the invention, but as merely providing illustrations of some of the presently preferred embodiments of this invention. Thus the scope of this invention should be determined by the appended claims and their legal equivalents. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims.

1-24. (canceled)

25. A method of evaluating an intervertebral disc space, comprising the steps of:
forming at least one annulotomy in an annulus;
removing through the annulotomy at least a portion of nucleus material in the intervertebral disc space to form a cavity;
delivering an evaluation material into the cavity;
setting the evaluation material to comprise a three-dimensional model generally corresponding to a shape of at least a portion of the cavity;
deforming the three-dimensional model a sufficient amount to be removed through an annulotomy while minimizing damage to any annular tissue surrounding the annulotomy;
removing the three-dimensional model from the cavity through the annulotomy;
permitting the three-dimensional model to substantially resume the shape of at least the portion of the cavity; and
evaluating the three-dimensional model to determine at least one geometric feature of the cavity.

26. The method of claim 25 comprising the step of removing additional nucleus material as necessary until the cavity comprises a desired geometry.

27. The method of claim 25 wherein the evaluation material comprises one of a low density polymer foam, a balloon containing a low density foam, a shaped memory polymer, a shaped memory alloy, a shape memory mesh framework, a shaped memory gel, or a wire loop.

28. The method of claim 25 comprising the steps of delivering a evaluation material into an expandable member located in the cavity.

29. The method of claim 28 wherein the expandable member comprises one of a balloon or a mesh structure.

30. The method of claim 28 comprising the step of deflating the expandable member before the step of removing the three-dimensional model from the cavity through the annulotomy.

31. The method of claim 25 comprising the steps of:
positioning a mesh structure in the cavity;
at least partially expanding the mesh structure in the cavity;
allowing the evaluation material to at least partially set; and
removing the evaluation material and the mesh from the cavity.

32. The method of claim 31 comprising the steps of:
engaging a distal end of a compression member with a distal portion of the mesh structure; and
delivering the evaluation material through the compression member.

33. The method of claim 31 comprising the steps of:
engaging a distal end of a compression member with a distal portion of the mesh structure; and
restraining the distal portion of the mesh structure while applying tension to the mesh structure.

34. The method of claim 25 wherein the step of delivering an evaluation material comprises the steps of:
positioning a balloon comprising a shaped memory material in the cavity;
inflating the balloon to an expanded configuration generally corresponding to a shape of the cavity; and
applying a stimulus to set the shape of the shape memory material.

35. The method of claim 34 wherein the stimulus comprises one or more of a heat source, a heat sink, an electromagnetic radiation generator, or a magnetic field generator.

36. The method of claim 34 comprising the steps of:
deflating the balloon;
removing the balloon from the cavity; and
inflating the balloon to the expanded configuration generally corresponding to a shape of the cavity.

37. The method of claim 25 comprising the steps of:
applying a force to the three-dimensional model in the cavity so that the three-dimensional model has a cross-sectional area generally corresponding to a cross-sectional area of the annulotomy; and
removing the three-dimensional model from the cavity through the annulotomy.

38. The method of claim 25 comprising the steps of:
introducing a removal tool into the cavity; and
removing the three-dimensional model from the cavity through the annulotomy.

39. The method of claim 25 comprising the steps of applying tension to the three-dimensional model during the step of removing.

40. The method of claim 25 comprising the steps of applying a torque to the three-dimensional model during the step of removing.

41. The method of claim 25 comprising the steps of deforming the three-dimensional model before the step of removing the three-dimensional model from the cavity.

42. The method of claim 25 comprising the step of locating a device between the three-dimensional model and at least a portion of the annulotomy during the removal step.

43. The method of claim 25 comprising the step of combining reinforcing fibers with the evaluation material.

44. The method of claim 25 comprising the steps of:
imaging the intervertebral disc space containing the three-dimensional model; and
evaluating a geometry of the three-dimensional model within the intervertebral disc space.

45. The method of claim 25 comprising the steps of:
imaging the intervertebral disc space;
estimating the volume of the cavity based on imaging; and
comparing the volume of the three-dimensional model with the estimated volume of the cavity.

46. The method of claim 25 comprising repeating the nucleus removing steps until the cavity is generally centered within the disc space.

47. The method of claim 25 comprising repeating the nucleus removing steps until the cavity is symmetrical relative to the midline of the spine.

48. The method of claim 25 comprising the steps of:
electronically scanning the three-dimensional model; and
generating a computer model of the three-dimensional model.

49. The method of claim 25 wherein the evaluation material is radiopaque.

50. The method of claim 25 comprising the step of removing at least a portion of the annulus before delivering the evaluation material.

51. The method of claim 25 comprising the step of substantially removing the annulus before delivering the evaluation material.

52. A method for evaluating an intervertebral disc space, comprising the steps of:
forming at least one annulotomy in an annulus;
removing through the annulotomy at least a portion of nucleus material in the intervertebral disc space to form a cavity;
delivering a shaped memory material into the cavity;
setting the shaped memory material to comprise a three-dimensional model generally corresponding to a shape of at least a portion of the cavity;
deforming the three-dimensional model a sufficient amount to be removed through an annulotomy while minimizing damage to any annular tissue surrounding the annulotomy;
removing the three-dimensional model from the cavity through the annulotomy;
permitting the three-dimensional model to substantially resume the shape of at least the portion of the cavity; and
evaluating the three-dimensional model to determine at least one geometric feature of the cavity.

53. The method of claim 52 wherein the shaped memory material comprises one of a shaped memory polymer, a shaped memory alloy, a shape memory mesh framework, a shaped memory gel, or a wire loop.

54. The method of claim 52 comprising the step of allowing the shaped memory material to set a sufficient amount to permit the three-dimensional model to be removed from the cavity.

55. The method of claim 52 comprising the step of delivering the shaped memory material into the cavity with a balloon.

56. The method of claim 55 comprising the step of deflating the balloon before the step of removing the three-dimensional model from the cavity through the annulotomy.

57. The method of claim 52 wherein the step of delivering the shaped memory material comprises the steps of:
positioning a balloon in the cavity with the shaped memory material; and
inflating the balloon and the shaped memory material to an expanded configuration generally corresponding to a shape of the cavity.

58. The method of claim 57 comprising the steps of:
deflating the balloon;
removing the balloon and the shaped memory material from the cavity; and
inflating the balloon and the shaped memory material to the expanded configuration generally corresponding to a shape of the cavity.

59. The method of claim 51 comprising the step of applying a stimulus to set the shape of the shape memory material.

60. The method of claim 51 comprising the steps of:
imaging the intervertebral disc space containing the three-dimensional model; and
evaluating a geometry of the three-dimensional model within the intervertebral disc space.

61. The method of claim 51 comprising the steps of:
imaging the intervertebral disc space;
estimating the volume of the cavity based on imaging; and
comparing the volume of the three-dimensional model with the estimated volume of the cavity.

62. The method of claim 51 comprising the steps of:
electronically scanning the three-dimensional model; and
generating a computer model of the three-dimensional model.

63. The method of claim 51 wherein the shaped memory material is radiopaque.
